(19) United States
(12) Patent Application Publication
(10) Pub. No.: US 2012/0142656 A1
(43) Pub. Date: Jun. 7, 2012

(54) BUCCAL APPLICATION SYSTEM COMPRISING 17A-ESTRADIOL

(75) Inventors: Thomas Gräser, Erfurt-Windischholzhausen (DE); Ralf Ladwig, Jena (DE); Abdul-Abbas Al-Mudhaffar, Jena (DE)

(73) Assignee: BAYER PHARMA AKTIENGESELLSCHAFT, Berlin (DE)

(21) Appl. No.: 13/148,300

(22) PCT Filed: Feb. 2, 2010
(86) PCT No.: PCT/EP2010/000619

§ 371 Date: Oct. 28, 2011

(30) Foreign Application Priority Data
Feb. 5, 2009 (DE) 10 2009 007 771.5

(51) Int. Cl. A61K 31/565; C07J 1/00; A61P 5/30

(52) U.S. Cl. 514/182; 552/630

(57) ABSTRACT

The invention relates to application systems for buccal application, comprising 17α-estradiol in a dosage of 50 to 400 μg, preferably 200 to 400 μg, particularly preferably 300 μg, together with one or more pharmaceutically acceptable additives or media for implementing the treatment or minimization of hot flashes in women having estrogen deficiencies.

### TECHNICAL FIELD

The invention relates to an application system for the buccal administration of 17α-estradiol for the treatment of hot flashes in women with natural (menopause) or surgically induced (ovarectomy) estrogen deficiency symptoms. The 17α-estradiol is used in a low dose of 50 to 400 μg, preferably 200 to 400 μg, particularly preferably 300 μg, together with one or more pharmaceutically acceptable excipients/vehicles.

### PRIOR ART

The substitution of estrogens in the case of appropriate deficiency symptoms (menopause, surgically induced menopause, GnRH treatment) is a well-established procedure with very good therapeutic success. A multiplicity of pharmacological preparations are employed for this, which mostly contain 17β-estradiol and its derivatives as estrogenic basic components. Just as widespread are preparations which use the so-called mare's urine estrogens as estrogen component. The latter are a mixture of a multiplicity of substances with differing estrogenic activity, which in total have a comparable effect on estrogen deficiency symptoms as the natural 17β-estradiol. In patients with an intact uterus, it is necessary to add a gestagen component for at least 12 days of a 28-day treatment cycle in order to prevent hypertrophy or degeneration of the endometrium.

“Classical” hormone replacement therapy (HRT) has come under criticism (Rossouw J E et al. Risks and benefits of estrogen plus progestin in healthy postmenopausal women: principal results from the Women's Health Initiative randomized controlled trial. J Am Med Assoc 2002; 288:321-33) after a large randomized, placebo-controlled study in the USA was carried out on more than 16,000 postmenopausal patients (Women's Health Initiative—WHI). In contrast to the positive effects shown before this study in many investigations, especially on the cardiovascular system, contrary findings were achieved in the WHI study. In addition, there was an increased risk of breast cancer in the group treated with hormones, which led to the premature discontinuation of the study. The study results have been carefully reanalyzed after the first publication and in some cases revised in their interpretation (Davey D A. Hormonal replacement therapy: time to move on? J Brit Menopause Soc 2006; 12:75-80). Nevertheless, the WHI study has led to a dramatic change in the prescription of HRT preparations and also to a considerable uncertainty of the users. There are therefore attempts to replace the “classical” hormone replacement therapy by new treatment methods.

17α-Estradiol is the naturally occurring epimer of 17β-estradiol and differs only in its stereochemistry on carbon atom 17. In humans, it is only detectable in very small concentrations and its physiological role is unclear. Markedly higher concentrations can be found in the animal kingdom, in particular in the urine of pregnant mares (Husmann F. Die Wirkungen von 17α-Östrogenen. 1. Teil: Grundlagen. [The actions of 17α-estrogens. 1st part: Fundamentals] horme 2003; 16:1-8). 17α-Estradiol is a constituent of mare's urine estrogens to approximately 3.7% (Washburn SA et al. Effects of 17α-dihydroequilenin sulfate on atherosclerotic male and female rhesus monkeys. Am J Obstet Gynaecol 1996; 175:341-51) and has been used in this form for about 50 years for the treatment of menopausal symptoms in humans (however as a mixture with a multiplicity of other substances with estrogenic action). It shows an approximately 5-fold weaker binding affinity to the estrogen receptor and an at least 200-times weaker action in the trans-activation assay than 17β-estradiol and is therefore to be characterized as a weak estrogen. Simultaneously, on the basis of its chemical structure, as also 17β-estradiol, it has a number of non-genomic actions, which have been taken into consideration for pharmaceutical development.

Surprisingly, it has been shown that 17α-estradiol is suitable as a monosubstance for the treatment of hot flashes both in postmenopausal women and younger patients after removal of both ovaries. This result was not obvious according to the state of scientific knowledge, since 17α-estradiol, as explained, is a weak estrogen and only has a low binding capacity to the estrogen receptor.

European patent EP 1 032 398 B1 is directed at the treatment of postmenopausal women using very low estrogen doses, such as, for example, less than 0.5 mg. The treatment of hot flashes is mentioned here.

European patent EP 1 539 184 B1 discloses an estrogen therapy comprising different doses in various phases.

A solid oral dose form is disclosed by WO 2006/048261, comprising an amount that is therapeutically equivalent to 0.01-0.5 mg of estradiol hemihydrate.

WO 2007/119151 discloses the reduction of menopausal symptoms in female patients, such as hot flashes, using a daily estradiol dose of 0.25-0.42 mg.

### DESCRIPTION OF THE INVENTION

The object of the present invention was to find a possibility for the treatment of hot flashes in women having natural or surgically induced estrogen deficiency symptoms.

The steroid hormone comprised in the appropriate application regime should be low-dose, released with a high bioavailability and the maximum blood level (=maximum concentration) should be achieved within at most one hour.

The object underlying the present invention was achieved by the provision of an application system for the buccal administration of 17α-estradiol for the treatment of hot flashes in women with natural (menopause) or surgically induced (ovarectomy) estrogen deficiency symptoms.

Here, the 17α-estradiol is used in a low dose of 50 to 400 μg, preferably 200 to 400 μg, particularly preferably 300 μg, together with one or more pharmaceutically acceptable excipients/vehicles.

The use of 17α-estradiol in a low dose, i.e. less than 500 μg, particularly less than 400 μg, for the treatment of (exclusively) vasomotor symptoms (hot flashes, episodes of sweating) is claimed. The mechanism of the favorable influencing of hot flashes is not mediated here by means of the classical genomic action and also not by means of antioxidative mechanisms, but by means of catechol estrogens (metabolites of estradiol), which stabilize the catecholamine degradation in the CNS (by means of catechol O-methyl transferase [COMT]) and thus counteract an adjustment of the body core temperature.

It is also known that 17α-estradiol has an extremely high first-pass metabolism in the liver, so that primarily it must be assumed from this to have to administer relatively high doses (at least 2 mg) on oral administration.

The invention surprisingly claims a buccal application form on the basis of a bioadhesive tablet. This bioadhesive tablet makes possible absorption of the active compound even in the oral cavity with a thereby increased bioavailability and avoidance of the hepatic first-pass effect, i.e. in the present patent application this leads to the treatment of hot flashes by means of 17α-estradiol in a low dose.

Gestagen addition can be dispensed with, so that possibly no undesired effects occur on the breast.

### Exemplary Embodiments

### Example 1

0.3 mg 17α-estradiol tablets having the following composition are prepared:

| Active substance layer [mg] | | Adhesive layer [mg] | |
|---|---|---|---|
| 17α-Estradiol | 0.3 | Mannitol | 17.4 |
| Mannitol | 75.291 | Cellactose | 34.82 |
| Cellactose | 50.209 | Carmellose sodium | 10.18 |
| Carmellose sodium | 1.08 | Iron oxide red | 0.05 |
| Magnesium stearate | 1.7 | Magnesium stearate | 0.65 |
| Talc | 5.07 | Talc | 1.9 |
| Sodium carboxymethylstarch | 1.35 | | |

All substances are mixed and granulated in a suitable manner and after the conclusion of the granulation process tableting is carried out.

### Example 2

Furthermore, 0.4 mg 17α-estradiol tablets having the following composition are prepared:

| Active substance layer [mg] | | Adhesive layer [mg] | |
|---|---|---|---|
| 17α-Estradiol | 0.4 | Mannitol | 17.4 |
| Mannitol | 75.191 | Cellactose | 34.82 |
| Cellactose | 50.209 | Carmellose sodium | 10.18 |
| Carmellose sodium | 1.08 | Iron oxide red | 0.05 |
| Magnesium stearate | 1.7 | Magnesium stearate | 0.65 |
| Talc | 5.07 | Talc | 1.9 |
| Sodium carboxymethylstarch | 1.35 | | |

All substances are mixed and granulated in a suitable manner and after the conclusion of the granulation process tableting is carried out.

### Investigations of the Activity

### Description of the Figures—Pharmacokinetics

The invention is described in more detail with reference to the attached figure in conjunction with Table 1.

| | 0.3 mg of 17α-estradiol | 0.4 mg of 17α-estradiol |
|---|---|---|
| | Concentration C | Concentration C |
| Time [h] | [pg/ml] | [pg/ml] |
| 1 | 280 | 315 |
| 2 | 122 | 134 |
| 3 | 63 | 64 |
| 4 | 42 | 46 |
| 6 | 22 | 27 |
| 8 | 16 | 20 |
| 12 | 10 | 15 |
| 18 | 6 | 9 |
| 24 | 5 | 7 |

Table 1 shows the data of the concentration of estradiol in pg/ml over the release period in hours = h

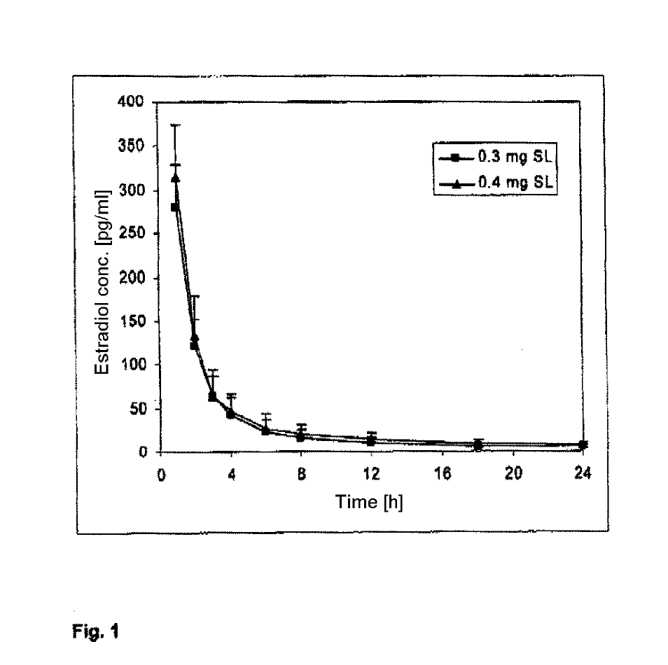

In FIG. 1, the plasma level course of 17α-estradiol (measured using GCMS) over time is shown, i.e. the concentration of the estradiol as a function of time after administration of 0.3 mg and 0.4 mg of 17α-estradiol buccal tablets in female subjects (n=6) is shown.

The buccal application system dissolves in the oral cavity preferably in a period of less than 180 min, particularly preferably in a period of less than 120 min. The 17α-estradiol entering directly into the blood circulation from the application system leads to a rapid increase in the concentration of 17α-estradiol in the blood. A maximum in the concentration of the 17α-estradiol in the blood is preferably achieved here in a period of less than 60 min—particularly preferably in a period between 30 and 60 min—after application. The high bioavailability of the 17α-estradiol and the absorption of the active substance even in the oral cavity is characteristic of the application system according to the invention, so that a first-pass effect is avoided and a high plasma level is achieved. A bioavailability of at least 80% can be achieved using the buccal application system. In a particularly preferred embodiment, the 17α-estradiol is released with a bioavailability of 70 to 90%.

### Clinical Investigation

In a double-blind, randomized clinical study in the crossover design, 20 postmenopausal or ovarectomized women were treated for 8 weeks either with 0.4 mg of 17α-estradiol in the form of a buccal tablet or placebo. Hot flashes were recorded before the beginning of treatment and on one of the last 3 days of treatment week 4. This was carried out according to subjective criteria (number and severity of the events) using patient diaries and objectively by continuous measurement of the skin temperature.

Moreover, the tolerability of the treatment regime was determined by monitoring of undesired events and standardized laboratory investigations.

In addition, the measurement of the endometrial thickness was carried out by transvaginal ultrasound investigations.

The administration of 17α-estradiol led to a rapid reduction of the number and intensity of hot flashes in the drug group, while only a moderate influence was to be observed in the placebo group. Before the beginning of treatment, the number of hot flashes per 24 h was 11.9±2.1 in the drug group and 12.3±1.9 in the placebo group. After treatment for 4 weeks, values of 3.1±1.8 resulted in the drug group and 7.7±4.1 in the placebo group. These values determined on the basis of the patient diaries showed a good correlation to the objectively measured changes in the skin temperature. The average increase during a hot flash assessed as severe by the patient was on average 2.3±1.4° C. The endometrial thickness measured by transvaginal ultrasound was not significantly altered in the two groups. The side-effect profile was comparable for both investigation groups.

1. An application system for the buccal administration comprising 17α-estradiol in a dose of 50 to 400 μg together with one or more pharmaceutically acceptable excipients/vehicles for the treatment of hot flashes in women with natural (menopause) or surgically induced (ovarectomy) estrogen deficiency symptoms.

2. The application system as claimed in claim 1, characterized in that 17α-estradiol is present at a dose of 200 to 400 μg.

3. The application system as claimed in claim 1, characterized in that 17α-estradiol is present at a dose of 300 μg.

4. The application system as claimed in claim 1, characterized in that the application system is formulated as a bioadhesive tablet.

5. A method of treating hot flashes in women, which are attributed to natural (menopause) or surgically induced (ovarectomy) estrogen deficiency, comprising the step of administering 17α-estradiol formulated as a buccal application system for bioadhesive administration in the form of a tablet, which contains 17α-estradiol in a dose of 50 to 400 μg.

6. The method of claim 5, characterized in that the tablet contains 17α-estradiol in a dose of 200 to 400 μg.

7. The method of claim 5, characterized in that the tablet contains 17α-estradiol in a dose of 300.

8. The method of claim 5, characterized in that the 17α-estradiol is formulated as an application system for once-daily administration.

9. Process for the production of a buccal delivery system comprising the step of formulating 17α-estradiol as a buccal application system for bioadhesive administration in the form of a tablet, which contains 17α-estradiol in a dose of 50 to 400 μg
for the treatment of hot flashes in women, which are to due to natural (menopause) or surgically induced (ovarectomy) estrogen deficiency.

10. The process of claim 9, characterized in that the tablet contains 17α-estradiol in a dose of 200 to 400 μg.

11. The process of claim 9, characterized in that the tablet contains 17α-estradiol in a dose of 300 μg.

12. The process of claim 9, characterized in that the 17α-estradiol is formulated as an application system for once-daily administration.
